(19) United States
(12) Patent Application Publication
(10) Pub. No.: US 2025/0204819 A1
(43) Pub. Date: Jun. 26, 2025

(54) CONTINUOUS BIOLOGICAL SENSOR WITH ENZYME IMMOBILIZATION

(71) Applicant: Allez Health Inc., Carlsbad, CA (US)

(72) Inventors: Mark Wu, San Diego, CA (US); Huashi Zhang, San Juan Capistrano, CA (US); Robert James Boock, Carlsbad, CA (US); Yubin Huang, Vista, CA (US); Qinyi Yan, San Diego, CA (US); Michael Christophe Walsh, Seattle, WA (US)

(73) Assignee: Allez Health Inc., Carlsbad, CA (US)

(21) Appl. No.: 19/047,285

(22) Filed: Feb. 6, 2025

Related U.S. Application Data
(63) Continuation of application No. 17/449,562, filed on Sep. 30, 2021.
(60) Provisional application No. 63/088,018, filed on Oct. 6, 2020.

(51) Int. Cl. A61B 5/145; A61B 5/1486; C12N 11/082

(52) U.S. Cl. CPC A61B 5/14532; A61B 5/1486; C12N 11/082

(57) ABSTRACT

A working wire of a continuous biological sensor is disclosed. The working wire includes a substrate for the sensor having a conductive surface. An enzyme layer is on the conductive surface and includes an enzyme, an immobilization matrix, and an enzyme immobilization network. The enzyme immobilization network is formed using a polymeric crosslinking agent and a non-polymeric crosslinking agent crosslinking the enzyme and the immobilization matrix. The polymeric crosslinking agent and the non-polymeric crosslinking agent are a combination of polyethylene glycol (PEG) dialdehyde and glutaraldehyde. A protective layer is over the enzyme layer.

### RELATED APPLICATIONS

This application is a continuation of U.S. Non-Provisional patent application Ser. No. 17/449,562, filed Sep. 30, 2021, and entitled “Working Wire for a Continuous Biological Sensor with an Enzyme Immobilization Network,” which claims priority to U.S. Provisional Patent Application No. 63/088,018 filed on Oct. 6, 2020, and entitled “Stabilized Enzymatic Sensor,” both of which are hereby incorporated by reference in full.

This application is related to U.S. Provisional Application 63/037,072 filed Jun. 10, 2020, and entitled “Sterilizable Metabolic Analyte Sensor,” which is incorporated herein as if set forth in its entirety. This application is also related to U.S. patent application Ser. No. 16/375,891, filed Apr. 5, 2019 and entitled “Continuous Glucose Monitoring Device”; which claims priority to (1) U.S. Provisional Application No. 62/653,821, filed Apr. 6, 2018, and entitled “Continuous Glucose Monitoring Device”; (2) U.S. Provisional Application No. 62/796,832, filed Jan. 25, 2019, and entitled “Carbon Working Electrode for a Continuous Biological Sensor”; and (3) U.S. Provisional Application No. 62/796,842, filed Jan. 25, 2019, and entitled “Enhanced Membrane Layers for the Working Electrode of a Continuous Biological Sensor”; each of which is incorporated herein as if set forth in their entirety.

### BACKGROUND

Medical patients often have diseases or conditions that require the measurement and reporting of biological conditions. For example, if a patient has diabetes, it is important that the patient have an accurate understanding of the level of glucose in their blood. Traditionally, diabetes patients monitor their glucose levels by pricking their finger with a small lancet, allowing a drop of blood to form, and then dipping a test strip into the blood. The test strip is positioned in a handheld meter that performs an analysis on the blood and visually reports the measured glucose level to the patient. Based upon this reported level, the patient makes important decisions on what food to consume, or how much insulin to inject into their blood. Although it would be advantageous for the patient to check glucose levels many times throughout the day, due to the pain and inconvenience of pricking, many patients fail to adequately monitor their glucose levels. As a result, the patient may cat improperly or inject either too much or too little insulin. Either way, the patient has a reduced quality of life and increased chance of causing permanent damage to their health and body. Diabetes is a devastating disease that if not properly controlled can lead to terrible physiological conditions such as kidney failure, skin ulcers, bleeding in the eyes, blindness, pain and the possible amputation of limbs.

Regular and accurate monitoring of glucose levels is critical for diabetes patients. To facilitate such monitoring, continuous glucose monitoring (CGM) sensors are a type of device in which glucose is automatically measured from fluid sampled just under the skin multiple times a day. CGM devices typically involve a small housing in which the electronics are located and which is adhered to the patient's skin to be worn for a period of time. A small needle within the device delivers the subcutaneous sensor which is often electrochemical. In this way, a patient may install a CGM sensor on their body, and the CGM sensor will provide automated and accurate glucose monitoring for many days without any action required from the patient or a caregiver. It will be understood that depending upon the patient's needs, continuous glucose monitoring may be performed at different intervals. For example, some continuous glucose monitors may be set or programmed to take multiple readings per minute, whereas in other cases, the continuous glucose monitor can be programmed or set to take readings every hour or so. It will be understood that a continuous glucose monitor may sense and report readings at different intervals.

Continuous glucose monitoring is a complicated process, and it is known that glucose levels in the body fluid can significantly rise/increase or lower/decrease quickly, due to several causes. Accordingly, a single glucose measurement provides only a snapshot of the instantaneous level of glucose in a patient's body. Such a single measurement provides little information about how the patient's use of glucose is changing over time, or how the patient reacts to specific dosages of insulin. Accordingly, even a patient that is adhering to a strict schedule of fingerstick testing will likely be making incorrect decisions as to diet, exercise, and insulin injection. Of course, this is exacerbated by a patient that is less consistent or inaccurately performs their strip testing. To give the patient a more complete understanding of their diabetic condition and to get a better therapeutic result, some diabetic patients are now using continuous glucose monitoring.

Electrochemical glucose sensors operate by using electrodes which typically detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is biased. The reference electrodes essentially complete the electron flow in the electrochemical circuit. Three-electrode (or three-pole) designs have a working electrode, a reference electrode and a counter electrode. The counter electrode replenishes ionic loss at the reference electrode and is part of an ionic circuit.

Conventional CGM systems typically use a working wire that uses a core of tantalum on which a thin layer of platinum is deposited. Tantalum is a relatively stiff material that is able to be pressed into the skin without bending, although an introducer needle may be used to facilitate insertion. Further, tantalum is inexpensive as compared to other materials such as platinum, which makes for an economical working wire. As is well known, an enzyme layer is deposited over the platinum layer, which is able to accept oxygen molecules and glucose molecules from the body fluid of the user. The key chemical processes for glucose detection occur within the enzyme membrane. Typically, the enzyme membrane has one or more glucose oxidase enzymes (GOx) dispersed within the enzyme membrane. When a molecule of glucose and a molecule of oxygen (O₂) are combined in the presence of the glucose oxidase, a molecule of gluconate and a molecule of hydrogen peroxide (H₂O₂) are formed. In one construction, the platinum surface facilitates a reaction wherein the hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which is intended to be related to the number of glucose molecules oxidized. A measurement of the electrical current on the platinum wire can thereby be associated with a particular level of glucose in the patient's body fluid such as blood or interstitial fluid.

The working wire is then associated with a reference electrode, and in some cases one or more counter electrodes, which form the CGM sensor. In operation, the CGM sensor is coupled to and cooperates with electronics in a small housing in which, for example, a processor, memory, a wireless radio, and a power supply are located. The CGM sensor typically has a disposable applicator device that uses a small introducer needle to deliver the CGM sensor subcutaneously into the patient. Once the CGM sensor is in place, the applicator is discarded, and the electronics housing is attached to the sensor. Although the electronics housing is reusable and may be used for extended periods, the CGM sensor and applicator need to be replaced quite often, usually every few days.

Unfortunately, conventional CGM sensors have a limited useful life, and therefore the patient or user must remove the old sensor and apply a new sensor to a new location on the body. This is not only inconvenient, but can be painful, and also increases the cost of using the CGM system. As the sensor is prone to damage during application, increased number of insertions means increased damaged sensors, and again, increased cost.

Limited stability of the enzyme layer is a key factor in the short useful life of the conventional CGM sensor. Stability has two components: first, the enzyme layer must be sufficiently sensitive to enable generation of an electrical signal capable of use by the senor's electronics, and second, the sensitivity level needs to be maintained for several days. Typical known sensors have good stability for about 5 days, but then begin to steadily lose sensitivity. Then, over the next few days, the CGM system may be able to adjust to the reduced sensitivity using algorithmic processes, and the user may even be directed to do one or more local calibrations to the reduced sensitivity. Each of these local calibrations requires the user to do a finger-prick blood glucose test and enter the result into the CGM's electronics to reset calibration factors. With a combination of algorithmic adjustment and local calibrations, the typical known sensor needs to be replaced about 10-14 days due to reduced sensitivity.

It is also important that the sensor be sterile when the user or patient inserts it into their body. Accordingly, the sensor is typically inserted into a sealed package after it is manufactured, and then sterilized. One of the most common methods of sterilization is to expose the sealed package to a sterilization gas, such as ethylene oxide, which is generally referred to as EtO. It will be appreciated that several other sterilization gases exist and may be used depending upon the specific application and environmental conditions. Unfortunately, sterilizing the sensor using a sterilization gas such as EtO results in reducing the stability and sensitivity of the manufactured sensor. Stated differently, the stability of the sensor is better prior to sterilization than after the sterilization has been completed. To address this issue, it is known to use an alternative sterilization process, such as high-powered e-beam sterilization process. However, the e-beam process can be more expensive, less reliable, and often damages any electronics or electronic components in the sealed sensor package.

### SUMMARY

Disclosed herein is a working wire of a continuous biological sensor. The working wire includes a substrate for the sensor having a conductive surface. An enzyme layer is on the conductive surface and includes an enzyme, an immobilization matrix, and an enzyme immobilization network. The enzyme immobilization network is formed using a polymeric crosslinking agent and a non-polymeric crosslinking agent crosslinking the enzyme and the immobilization matrix. The polymeric crosslinking agent and the non-polymeric crosslinking agent are a combination of polyethylene glycol (PEG) dialdehyde and glutaraldehyde. A protective layer is over the enzyme layer.

Disclosed herein is a method of manufacturing a working wire of a continuous a biological sensor. The method includes providing a substrate for the sensor. The substrate has a conductive surface. An enzyme layer is formed on the conductive surface. The forming includes combining an enzyme with a solvent creating an enzyme mixture, mixing an immobilization matrix with the enzyme mixture, and combining a polymeric crosslinking agent and a non-polymeric crosslinking agent with the enzyme mixture and the immobilization matrix creating a crosslinked mixture. The polymeric crosslinking agent and the non-polymeric crosslinking agent are a combination of polyethylene glycol (PEG) dialdehyde and glutaraldehyde, to form an enzyme immobilization network that crosslinks the enzyme and the immobilization matrix. The crosslinked mixture is applied to the substrate. After the applying, the crosslinked mixture is cured on the substrate. A protective layer is formed over the enzyme layer.

### BRIEF DESCRIPTION OF THE DRAWINGS

Objects and advantages of the present disclosure will become apparent upon reading the following detailed description and upon referring to the drawings and claims.

FIG. 1A is a perspective view of a continuous glucose monitor, in accordance with some embodiments.

FIG. 1B is a partial schematic of the interior components of the continuous glucose monitor system with the cover and the base removed, in accordance with some embodiments.

FIG. 2 is a not-to-scale cross-sectional view of a working wire for a glucose-specific sensor, in accordance with some embodiments.

FIG. 3 is a not-to-scale cross-sectional diagram of a glucose-specific sensor for a continuous glucose monitor, in accordance with some embodiments.

FIG. 4 is a flowchart of a method for making a working wire for a continuous biological sensor, in accordance with some embodiments.

FIG. 5A is a graph showing sensitivity results for a continuous glucose monitor, in accordance with some embodiments.

FIG. 5B is a chart showing sensitivity results for sensors, in accordance with some embodiments.

FIG. 5C is a graph showing sensitivity results for a continuous glucose monitor, in accordance with some embodiments.

FIG. 6 is a graph showing sensitivity results for a continuous glucose monitor, in accordance with some embodiments.

### DETAILED DESCRIPTION

Described herein is a working wire for a continuous biological sensor such as a continuous glucose monitor, having an enzyme layer. The enzyme layer is formulated and processed to have an enzyme immobilization network. This enzyme immobilization network stabilizes the sensitivity of the sensor for an extended number of days, thereby increasing its useful life, and reducing the need for algorithmic corrections or local patient calibrations. The enzyme immobilization network has been observed and tested in accordance with the present disclosure to show an increase in the stabilization after sterilization with a sterilization gas such as EtO. In this way, the sensor having the enzyme immobilization network has extended stability and useful life after manufacture and exhibits better stability and a longer useful life after EtO sterilization. It will be appreciated that other sterilization gases may be used.

The enzyme immobilization network acts as an immobilization network for the metabolic biological enzyme, such as glucose oxidase enzymes (GOx). It will be appreciated that other enzymes may be used depending upon the particular metabolic analyte that is to be detected. For example, the enzyme lactate oxidase may be used to monitor lactic acid as the analyte, or the enzyme hydroxybutyrate dehydrogenase may be used to monitor ketone. This enzyme immobilization network may be formulated using either polymers or proteins. To create the enzyme immobilization network, these polymers or proteins are stabilized with the enzymes using crosslinking agents, such as polymeric or non-polymeric crosslinking agents. Once the sensor has been manufactured using such an enzyme immobilization network, the sensor exhibits dramatically improved stability, and exhibits increased stability after gas sterilization.

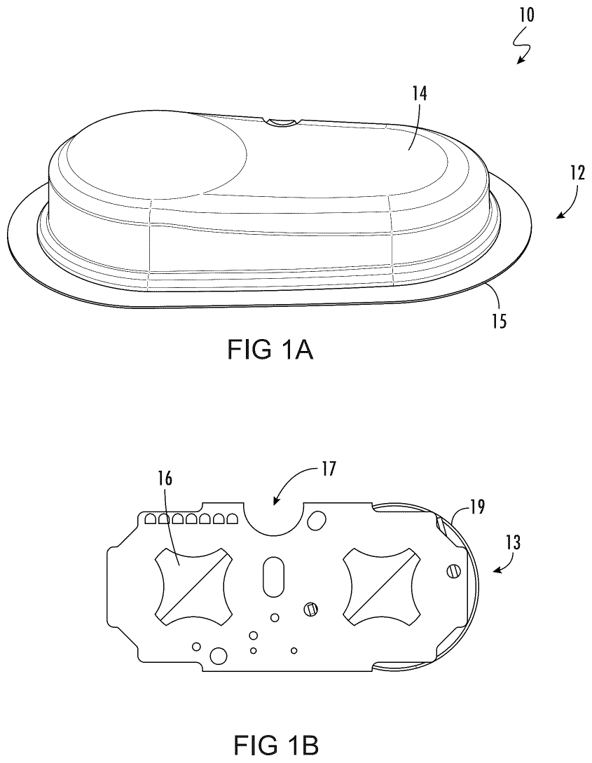

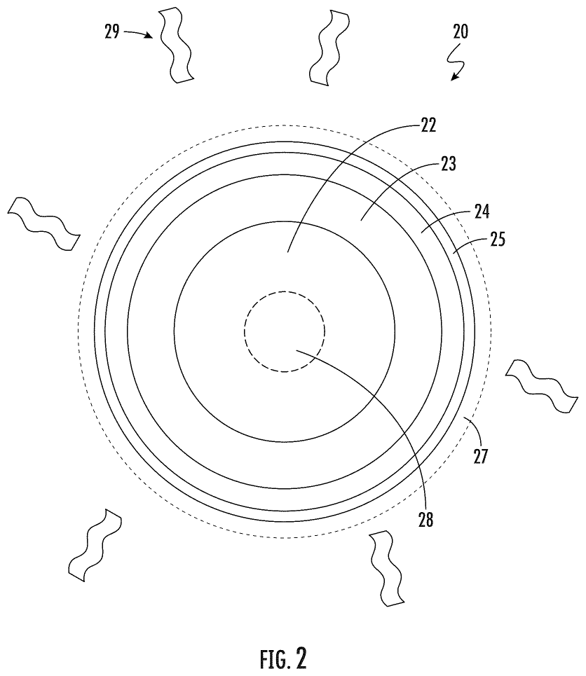

FIG. 1A is a perspective view of a continuous glucose monitor 10, in accordance with some embodiments. The continuous glucose monitor 10 has a package 12 which holds internal components 13 (see FIG. 1B). Package 12 has a cover 14 that sealably connects to a base 15 to provide a hermetic seal. In use, a patient or caregiver receives the package 12, and removes the cover 14 from its associated base 15. The patient or caregiver disposes of the cover 14, and adheres the base 15 to the patient, typically by means of an adhesive. FIG. 1B is a partial schematic of the interior components of the continuous glucose monitor 10 with the cover 14 and the base 15 removed, in accordance with some embodiments. Once the cover 14 and the base 15 have been removed from the package 12, the internal components 13 of the continuous glucose monitor 10 are exposed. These internal components 13 include an applicator 16, a continuous glucose monitor (CGM) sensor 17, and supporting electronics 19 that include a processor, components, and in some cases, a battery and a wireless radio. It will be appreciated that other structures may be provided, such as an inserter needle. With the base 15 adhesively attached to the patient's body, the patient or the caregiver engages the applicator 16 to insert the CGM sensor 17 under the skin of the patient. Once the CGM sensor 17 is fully inserted, the applicator 16 is released and in many cases may also be discarded. The patient now has an operating continuous glucose monitor 10 installed on their body, such that the CGM sensor 17 is inserted subcutaneously, and the electronics 19 are able to monitor glucose levels. In some embodiments, the electronics 19 also include a wireless radio for communicating results and alarms to a device, such as a BLUETOOTH® enabled mobile phone. In other embodiments, a radio may be provided separately from the electronics 19.

For the safety of the patient, it is critically important that the CGM sensor 17 be sterile at the time of insertion into the patient. As such, the entire package 12 is sterilized by the continuous glucose monitor manufacturer prior to shipping for patient use. For most efficient manufacturing, the continuous glucose monitor 10 is assembled in a clean, but not sterile environment. Accordingly, the CGM sensor 17, electronics 19 and applicator 16 are assembled onto the base 15, and then the cover 14 is sealed against the base 15. The package 12, which holds all the internal components 13, is then required to go under rigorous sterilization.

In known, typical sterilization processes for CGM sensors, the CGM sensor is first sterilized using electron beam sterilization (EBS), and at a later time, the electronics are connected to the CGM sensor, for example, after the CGM sensor has been inserted into the patient's body. However, EBS cannot be used for the continuous glucose monitor 10. In continuous glucose monitor 10, the CGM sensor 17 and the electronics 19 are manufactured and connected together prior to sterilization, and therefore any EBS of package 12 will destroy the electronics 19.

In embodiments of the present disclosure, the package 12 is sterilized using a gas sterilization process, such as by using EtO gas, where the continuous glucose monitor 10 is designed such that the electronics 19 are protected during sterilization. In conventional CGM system designs, EtO gas is effective in sterilizing the package 12, including the CGM sensor 17, but EtO gas is well known to negatively affect the performance of the CGM sensor by dramatically reducing the sensitivity and stability of the enzyme layer. The EtO gas, which can permeate deep into package 12 and into the CGM sensor 17, may damage the enzyme layer of CGM sensor 17. However, as will be described below in accordance with the present disclosure, CGM sensor 17 is particularly constructed to resist the negative effects of the EtO gas. As a result of protecting the enzymes in CGM sensor 17, package 12 may be efficiently and effectively sterilized using a gas sterilization process, including EtO gas. This protection for CGM sensor 17 is formulated to not only resist the negative effects of gas sterilization, but may actually increase the sensitivity and stabilization of the CGM sensor 17, resulting in a superior sensor. By protecting the enzymes and improving stability during gas sterilization, using EtO gas sterilization may even be considered the preferred process, even if electronics were not present during sterilization.

The gas sterilization process results in safe sterilization of a package containing both the CGM sensor 17 and the electronics 19, and may improve the stability and/or sensitivity of the enzyme layer for a better performing and longer lasting sensor. As a result of the efficient sterilization process for the continuous glucose monitor 10, as well as the improved performance of the CGM sensor 17, a far more cost-effective continuous glucose monitor 10 may be provided to the patient. Although the sterilization process is described in particular using EtO gas, it will be appreciated that other gases may be used, such as nitrogen dioxide, vaporized peracetic acid or hydrogen peroxide. It will be understood that other sterilization gases may be substituted according to application-specific requirements. Also, although the gas sterilization process is described in this disclosure as using EtO gas, it will be understood that the inventive principles extend to other gases and sterilization processes. In some embodiments, the CGM sensor can be packaged alone and subjected to e-beam sterilization, where the membrane layers of the sensor are configured to improve the stability and/or sensitivity of the sensor after e-beam sterilization compared to before sterilization. In some embodiments, the interference layer and/or the enzyme layer of a continuous glucose monitor are configured such that the continuous glucose monitor 10 has a performance characteristic that has a level that remains the same or is improved after completion of a sterilization process compared to before the sterilization process, where the sterilization can be gas or e-beam.

FIG. 2 depicts a not-to-scale cross-sectional view of a working wire 20 for a continuous glucose-specific sensor, in accordance with some embodiments. The working wire 20 is constructed with a substrate 22, which may be, for example tantalum. It will be appreciated that other substrates may be used, such as a Cr—Co alloy as set forth in co-pending U.S. Provisional patent application Ser. No. 17/302,415 entitled “Working Wire for a Biological Sensor” and filed on May 3, 2021; or a plastic substrate with a carbon compound as set forth in in co-pending U.S. patent application Ser. No. 16/375,887 entitled “A Carbon Working Electrode for a Continuous Biological Sensor” and filed on Apr. 5, 2019; all of which are hereby incorporated by reference. It will be appreciated that other substrate materials may be used. In general, the substrate 22 has an electrically conductive surface (i.e., outer surface) that is a conductive material. The conductive surface may be a metal, and may include platinum, platinum/iridium alloy, platinum black, gold or alloys thereof, palladium or alloys thereof, nickel or alloys thereof, titanium and alloys thereof. The conductive surface may include carbon in different forms, such as one or more carbon allotropes including nanotubes, fullerenes, graphene and/or graphite. The conductive surface may also include a carbon material such as diamagnetic graphite, pyrolytic graphite, pyrolytic carbon, carbon black, carbon paste, or carbon ink. In the embodiment of FIG. 2, the substrate 22 has a continuous layer 23 which is an outer surface of the substrate that is an electrically conductive. In this embodiment, the continuous layer 23 shall be described as platinum, although other conductive materials may be used as described throughout this disclosure. This platinum layer may be provided through an electroplating or depositing process, or in some cases may be formed using a drawn filled tube (DTF) process. It will be appreciated that other processes may be used to apply the platinum continuous layer 23.

The substrate 22, platinum continuous layer 23, interference layer 24, enzyme layer 25 and glucose limiting layer 27 form the key aspects of working wire 20. It will be understood that several other layers may be added depending upon the particular biologic being tested for, and application-specific requirements. In some embodiments, the substrate 22 may have a core 28. For example, if the substrate 22 is made from tantalum, a core of titanium or titanium alloy may be provided to provide additional strength and straightness. Other substrate materials may use other materials for its core 28.

In some embodiments, an interference layer 24 is applied over the platinum continuous layer 23. This interference layer, which will be fully described below, fully encases the platinum continuous layer 23, and is set between the platinum continuous layer 23 of the conductive surface and the enzyme layer 25. This interference layer is constructed to fully wrap the platinum layer, thereby protecting the platinum from further oxidation effects. The interference layer is also constructed to substantially restrict the passage of larger interferent contaminant molecules, such as acetaminophen, to reduce unwanted reactive species that can reach the platinum and skew the electrical signal results. Further, the interference layer is able to pass a controlled level of hydrogen peroxide (H₂O₂) from the enzyme layer to the platinum layer, thereby increasing sensitivity, stability and accuracy. A highly stable enzyme layer 25 is then applied, and finally a glucose limiting layer 27 is layered on top of the enzyme layer 25. This glucose limiting layer 27, such as glucose limiting layer described in co-pending U.S. patent application Ser. No. 16/375,877, may limit the number of glucose molecules that can pass through the glucose limiting layer 27 and into the enzyme layer 25.

If the sensor is a glucose sensor, then enzyme layer 25 most often uses GOx as the active enzyme, although it will be appreciated that other enzymes may be used, for example when biological substances other than glucose are being measured. For the sensor with working wire 20, the enzyme layer 25 is formulated to not only reduce any negative effects from sterilization, for example from exposure to EtO gas 29, but in some cases may be formulated such that the sterilization process actually improves the stability or sensitivity of the sensor. As will be more fully described below, the enzyme layer 25 may be formulated and processed with particular proteins or polymers, which enable improved sterilization response for the sensor with working wire 20.

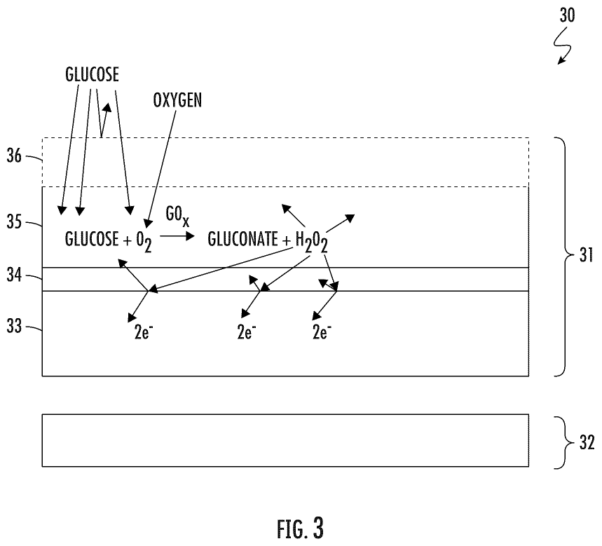

FIG. 3 is a not-to-scale cross-sectional diagram of a glucose-specific sensor for a continuous glucose monitor, in accordance with some embodiments. A sensor 30 is described in terms of a glucose monitor, but as with other embodiments in this disclosure, sensor 30 can also apply to the monitoring of other metabolites such as ketones or fatty acids. The sensor 30 has a working electrode 31 which cooperates with a reference electrode 32 (which, in some embodiments, may be constructed of a silver or a silver chloride) to provide an electrochemical reaction that can be used to determine glucose levels in the body fluid of a patient. Although sensor 30 is illustrated with one working electrode 31 and one reference electrode 32, it will be understood that some alternative sensors may use multiple working electrodes, multiple reference electrodes, and counter electrodes. It will also be understood that sensor 30 may have different physical relationships between the working electrode 31 and the reference electrode 32. For example, the working electrode 31 and the reference electrode 32 may be arranged in layers, spiraled, arranged concentrically, or side-by-side. It will be understood that many other physical arrangements may be consistent with the disclosures herein.

The working electrode 31 has a conductive portion, which is illustrated for sensor 30 as conductive wire 33. This conductive wire 33 can be for example, solid platinum, a platinum coating on a less expensive metal, carbon or plastic. In other words, conductive wire 33 may be a conductive surface (i.e., conducting layer) of a wire in some embodiments. It will be understood that other electron conductors may be used consistent with this disclosure. The working electrode 31 has a glucose limiting layer 36, which may be used to limit contaminations and the amount of glucose that is received into the enzyme membrane 35 (also called enzyme layer).

In operation, the glucose limiting layer 36 substantially limits the amount of glucose that can reach the enzyme membrane 35, for example only allowing about 1 of 1000 glucose molecules to pass. By strictly limiting the amount of glucose that can reach the enzyme membrane 35, linearity of the overall response is improved. The glucose limiting layer 36 also permits oxygen to travel to the enzyme membrane 35. The key chemical processes for glucose detection occur within the enzyme membrane 35. Typically, the enzyme membrane 35 has one or more glucose oxidase enzymes (GOx) dispersed within the enzyme membrane 35. When a molecule of glucose and a molecule of oxygen (O₂) are combined in the presence of the glucose oxidase, a molecule of gluconate and a molecule of hydrogen peroxide are formed. The hydrogen peroxide then generally disperses both within the enzyme membrane 35 and into interference membrane 34 (which may also be referred to in this disclosure as an interference layer). In sensor 30, the enzyme membrane 35 is stabilized by providing an enzyme immobilization network. In general embodiments, the enzyme immobilization network has molecules that are crosslinked to provide for the enhanced enzyme stabilization. For example, a working wire for a continuous biological sensor such as a continuous glucose monitor includes a substrate having a conductive surface and an enzyme layer formed on the conductive surface. The enzyme layer has a biological enzyme and a crosslinking agent, such as a polymeric and/or a non-polymeric crosslinking agent, crosslinking the enzymes and the immobilization matrix creating an enzyme immobilization network. In some embodiments, immobilization molecules form the matrix around the enzymes. A protective layer is included on the enzyme layer.

Two specific types of enzyme immobilization networks will be described. The first type of stabilized network uses a polymer-based immobilization matrix, such as one or more selected from polyurethane (PU), polyacrylic acid, polyacrylamide, polyvinylpyrrolidone (PVP), polyethylene glycol (PEG), or polyvinyl alcohol (PA) and its copolymers, or copolymers of N-(2-hydroxypropyl)-methacrylamide, polydimethylsiloxane (PDMS), polyamides, polyacrylates, polyethylene, polycarbonates or combinations thereof. In some embodiments, the immobilization network comprises crosslinked molecules of the polymer selected from polyurethane (PU), polyvinylpyrrolidone (PVP), or polyethylene glycol (PEG), or combinations thereof. For example, PVP may be used to thicken the material to enable dip coating, improve mobility for enhancing activity such as the enzyme reaction with glucose, and improve the enzyme layer glucose sensitivity. The second type of stabilized network uses a protein-based immobilization matrix, such as one or more selected from bovine serum albumin (BSA), human serum albumin (HSA), carboxymethyl cellulose (CMC), collagen or combinations thereof.

The selected immobilization matrix, whether polymers or proteins, are then immobilized into the enzyme immobilization network using a crosslinking agent. The crosslinking agent may a polymeric crosslinking agent, a non-polymeric crosslinking agent, or a combination of the polymeric crosslinking agent and the non-polymeric crosslinking agent. Examples of non-polymeric crosslinking agents may be selected from glutaraldehyde (GA), polyfunctional aziridine, bifunctional carbodiimide, dicyclohexyl carbodiimide, 1-ethyl-3-(3-dimethylaminopropyl) carbodiimide, N-hydroxysuccinimide, N-hydroxysulfosuccinimide, ethylene glycol bis(succinimidyl succinate) (EGS), ethylene glycol bis(sulfosuccinimidyl succinate) (SEGS), tris-(succinimidyl) aminotriacetate (TSAT), dimethyl pimelimidate (DMP), dimethyl suberimidate (DMS), 1,5-difluoro-2,4-dinitrobenzene (DFDNB), dimethyl 3,3′-dithiobispropionimidate (DTBP), NHS-Phosphine, NHS-PEG-azide, NHS-azide or combinations thereof. For example, more that one of these agents can be used together.

In some embodiments, the non-polymeric crosslinking agent may be selected from glutaraldehyde (GA), bifunctional carbodiimide, or combinations thereof. Glutaraldehyde may have an extremely strong effect on the enzyme layer and may be used in small amounts. For example, the ratio of enzyme to glutaraldehyde may be 80 to 1 or 75-82 to 1. Bifunctional carbodiimide may also be combined in small amounts such as 1% of total solution, or 0.8% to 1.5% of total solution.

Some embodiments may include water-soluble polymeric crosslinking agents selected from polyethylene glycol (PEG) dialdehyde, bifunctional PEG carbodiimide, PEGylated bis(sulfosuccinimidyl) suberate or combinations thereof. In some embodiments, large crosslinkers (e.g., high molecular weight) may be used. These water-soluble crosslinkers effectively wrap the GOx enzyme inside its chain to protect the GOx enzyme from contaminants. In some embodiments, polyvinylpyrrolidone (PVP) and an aqueous polyurethane dispersion solution were dissolved in water and mixed with GOx.

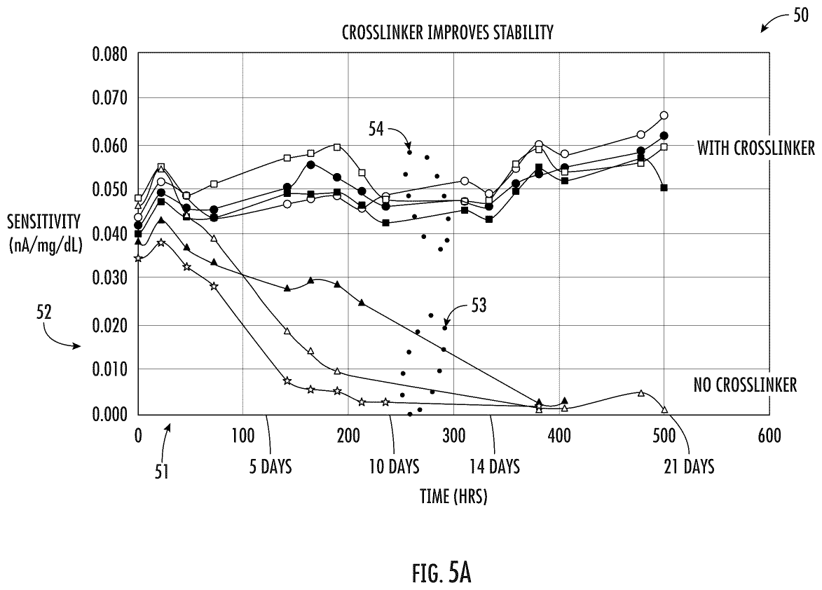

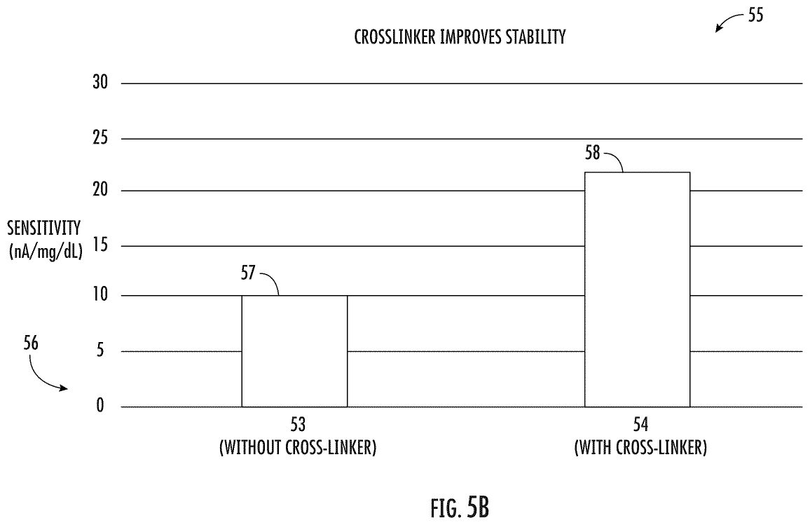

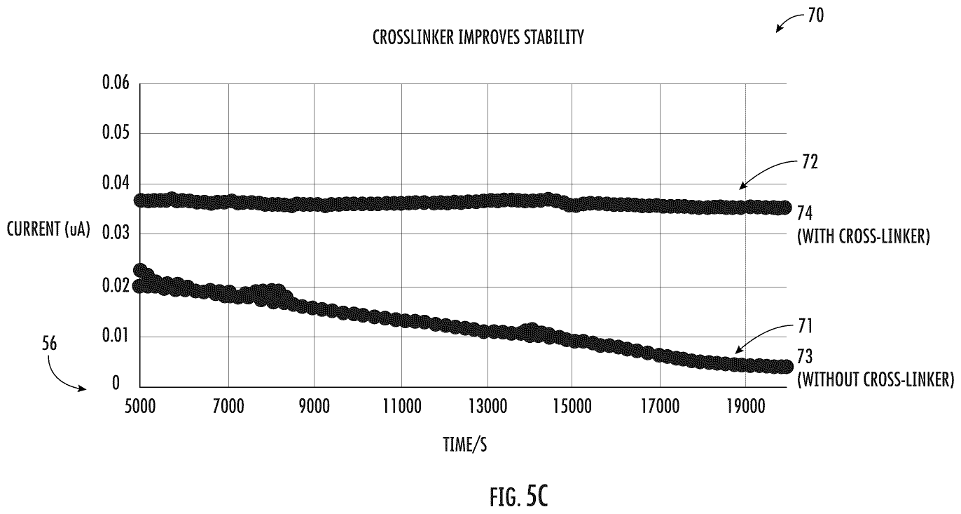

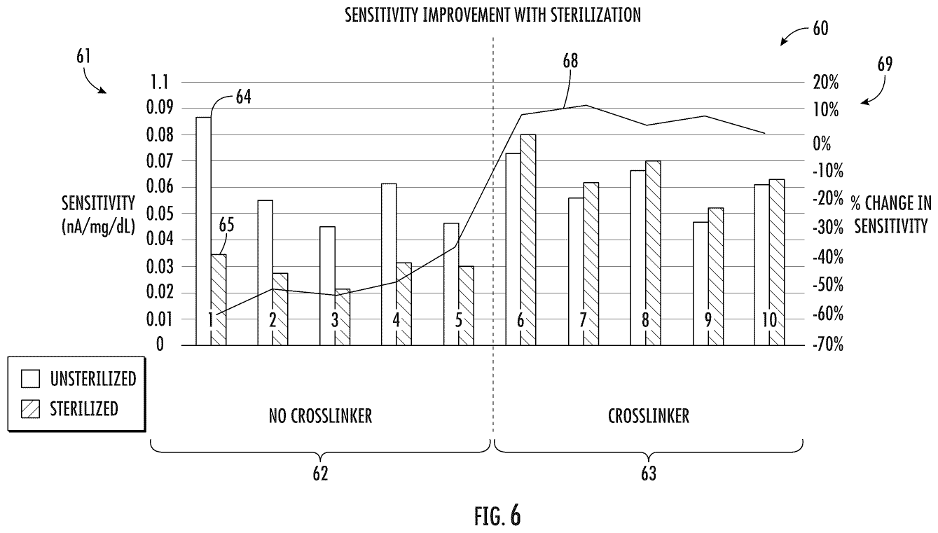

In some embodiments, the polymeric and non-polymeric crosslinking agents may be used together, such as polyethylene glycol (PEG) dialdehyde for the polymeric crosslinking agent and glutaraldehyde for the non-polymeric crosslinking agent. For example, the water-soluble polymeric crosslinking agent, such as polyethylene glycol (PEG) dialdehyde along with the non-polymeric crosslinking agent glutaraldehyde, is crosslinked with the enzyme as well as the immobilized matrix such as the polymer or protein. The crosslinking agents stabilize the enzymes, keeping the enzymes in place such as in the enzyme layer. In turn, there is little to no loss of glucose sensitivity over time. For example, during and after the process of gas sterilization such as by using EtO gas, there is little to no loss of glucose sensitivity. Data and the results of testing are discussed herein and presented in FIGS. 5A-7. In contrast, in conventional methods, the enzyme is not crosslinked to the enzyme nor to immobilized matrix (or molecules) so the enzyme is mobile and exhibits movement. For example, in conventional methods, the enzyme may be bound in polyurethane. In these systems, the outer layers such as the interference layer or glucose limiting layer only “traps” the enzyme in the enzyme layer but the enzyme is still free to move about in the layer. Moreover, in the embodiments disclosed herein, the crosslinkers stabilize the enzyme while still allowing them to be functional. For example, glutaraldehyde immobilizes the enzyme but by using polyethylene glycol (PEG) dialdehyde as “spacers,” it allows the enzymes to rotate around the crosslinked bonds. Thus, a balance is achieved between the stability while still enabling the enzyme to react with glucose.

In some embodiments, the crosslinking agents may be a combination of polymeric and non-polymeric crosslinking agents, and may be selected from polyethylene glycol (PEG) dialdehyde, bifunctional PEG carbodiimide, PEGylated bis(sulfosuccinimidyl) suberate, glutaraldehyde (GA), polyfunctional aziridine, bifunctional carbodiimide, dicyclohexyl carbodiimide, 1-ethyl-3-(3-dimethylaminopropyl) carbodiimide, N-hydroxysuccinimide, N-hydroxysulfosuccinimide, ethylene glycol bis(succinimidyl succinate) (EGS), ethylene glycol bis(sulfosuccinimidyl succinate) (SEGS), tris-(succinimidyl) aminotriacetate (TSAT), dimethyl pimelimidate (DMP), dimethyl suberimidate (DMS), 1,5-difluoro-2,4-dinitrobenzene (DFDNB), dimethyl 3,3′-dithiobispropionimidate (DTBP), NHS-Phosphine, NHS-PEG-azide, NHS-azide, or combinations thereof. The proportions of crosslinking agents in the mixture can be 10% to 90% for one crosslinking agent or combinations of crosslinking agents.

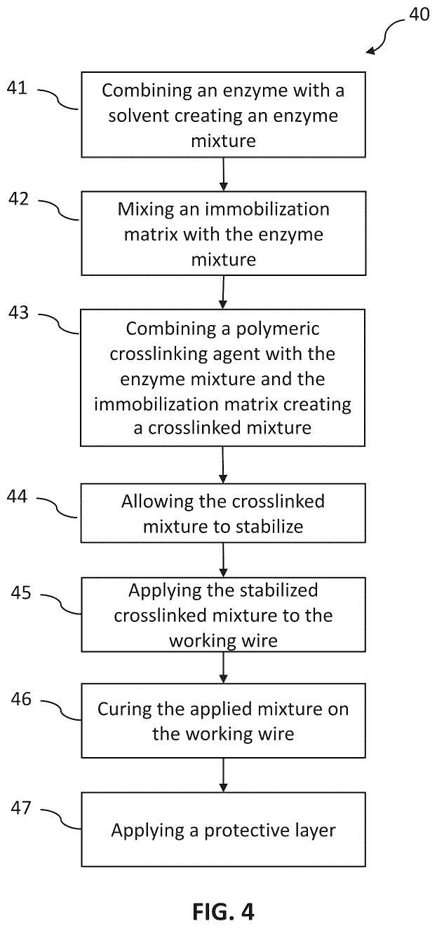

FIG. 4 is a flowchart of a method for making a working wire for a continuous biological sensor, in accordance with some embodiments. A method 40 of making a working wire for a continuous biological sensor includes an enzyme layer having an immobilization network. In one example, method 40 is used to make enzyme membrane 35 as described with reference to FIG. 3. As will be described below in accordance with the present disclosure, a method 40 for making a working wire for a continuous biological sensor such as a continuous glucose monitor, includes combining an enzyme with a solvent creating an enzyme mixture. An immobilization matrix is mixed with the enzyme mixture. After the mixing, a polymeric crosslinking agent is combined with the enzyme mixture and the immobilization matrix creating a crosslinked mixture. The crosslinked mixture is allowed to stabilize. The stabilized crosslinked mixture is applied to the working wire, and the applied mixture is cured on the working wire.

As illustrated at block 41, an enzyme formula is made by mixing an enzyme with a solvent creating an enzyme mixture. An appropriate solvent is selected, such as water for making a dip bowl enzyme formula. It will be appreciated that other solvents may be used depending upon the specific enzyme, polymer, protein, or crosslinking agent used. The particular enzyme is selected, such as GOx, when the sensor is intended to detect glucose. It will be understood that other enzymes will be selected for other types of analyte detections, such as lactate oxidase for monitoring lactic acid, or hydroxybutyrate dehydrogenase for monitoring ketone. At block 42, the enzyme is combined or mixed with an immobilization matrix, the immobilization matrix being the polymer, if a polymer-based stabilization network has been selected, or the enzyme is mixed with the protein, if a protein-based stabilization network has been selected. Immobilization molecules may form a matrix around the enzymes.

At block 43, after the mixing, a crosslinking agent is mixed into the enzyme mixture and immobilization matrix creating a crosslinked mixture. For example, once the enzyme has been fully mixed with the selected molecules, the crosslinking agent is then combined into the mixture, creating the crosslinked mixture. The crosslinking agent may be a polymeric crosslinking agent, a non-polymeric crosslinking agent, or a combination thereof. In some embodiments, the crosslinking agent is a polymeric crosslinking agent. The combining may further comprise combining a non-polymeric crosslinking agent with the enzyme mixture and the immobilization matrix creating a crosslinked mixture. At block 44, the crosslinked mixture is allowed to stabilize. For example, once the crosslinking agent or crosslinking agents have been thoroughly mixed into the formula, the formula is allowed to stabilize into a steady state. This may be indicated by no further significant viscosity change over time, enabling the crosslinking agent or agents to cooperate with the enzymes and molecules to form the enzyme immobilization network.

In some embodiments, the combining or mixing is performed by high shear mixing due to high concentrations of crosslinkers that exceeds 10% by weight. Crosslinking agents have fast reaction rates and will react with the nearest active site which leads to uneven crosslinking. Uneven distribution of crosslinking leads to an un-stabilize network and performance over time. High shear mixing creates a homogeneous solution with a uniform dispersion by adding energy to the system to redistribute the surfactant or crosslinking agent such as polyethylene glycol (PEG) dialdehyde, across the added materials. In some embodiments, other mixing techniques may be used such as stirring or impeller.

At block 45, the stabilized crosslinked mixture is applied to the working wire. For example, once the crosslink enzyme formula has stabilized, it may then be used in the manufacturing process to coat a sensor wire. The sensor wire will have a conductive substrate which has already been coated with an interference membrane. In this way, the stabilized crosslinked mixture is applied to the interference membrane, although it will be understood that other arrangements could be made. In some embodiments, the sensor wire is dipped into a vessel holding the stabilized crosslinked mixture. Other techniques for applying the enzyme layer to the wire may include, for example, spraying or printing. The working electrode may be dipped or submerged into the stabilized crosslinked mixture.

In some embodiments of block 45, the working electrode is held in the enzyme formula for a period of time, such as 10 to 60 seconds. It will be understood that several factors affect the thickness of the stabilized crosslinked mixture that adheres to the working wire. For example, factors include the rate at which the working wire is lowered into the stabilized crosslinked mixture, the amount of time the working wire is submerged in the stabilized crosslinked mixture, the rate at which the working wire is removed from the stabilized crosslinked mixture, environmental conditions like temperature, humidity, airflow during the dipping process, and straightness of the sensor wire. Further, aspects of the stabilized crosslinked mixture itself, such as temperature, viscosity, evaporation, homogeneity, and any movement due to mixing, also affect the thickness of the applied enzyme layer.

Additionally, the dipping or submerging may be done once, or may be repeated as needed to obtain sufficient absorption of the GOx to the desired depth and concentration. In some cases, the manufacturing processes will have a predefined target thickness for the enzyme layer. In such a circumstance, the manufacturing process will have a measuring process to determine the thickness after each dip, and then continue dipping the working wire until the target thickness has been reached. At block 46, the stabilized crosslinked mixture is cured on the working wire. For example, once a target thickness has been reached, the working wire is cured. The curing may involve, for example, drying the enzyme layer at an elevated temperature (e.g., at approximately 40° C. to 60° C., such as 50° C.). This curing process further stabilizes the enzyme immobilization network, thereby further increasing the overall stabilization for the enzyme layer. At block 47, a protective layer may be applied. For example, once the enzyme layer has been fully cured, the working wire may move to the next manufacturing process, which typically adds a protective layer or membrane around the enzyme layer. In some cases, this protective layer may be a glucose limiting layer, and in other cases it may be a bio-protective layer. It will be appreciated that other types of protective layers may encapsulate the enzyme layer.

The enzyme immobilization network acts as a wrap or shield to protect the GOx or other enzyme molecule, or to reduce the tendency of the enzyme to migrate within the enzyme layer. In some cases, the immobilization network may also act as a sacrificial barrier to interact with other molecules, such as the EtO gas, rather than having the EtO gas interact with and produce negative effects on the enzyme itself. For example, when proteins are used in the immobilization network, the EtO gas may first react with the protein where it is uniform across the layer. This diminishes the effect of EtO gas on the enzyme.

FIG. 5A is a graph showing sensitivity results for a continuous glucose monitor, in accordance with some embodiments. A graph 50 shows actual results of the improvement to the stability due to the enzyme immobilization network in non-sterilized sensors. All of the sensors were stored together at ambient room temperature until they were selected for testing. The graph 50 depicts a signal response of active sensors in-vitro/bench to mimic real life performance over 21 days. The sensors were place in a glucose solution resulting in the enzyme layer generating hydrogen peroxide (H₂O₂) that produces an electrochemical response. This was measured as a signal change to determine the performance of the sensor. The graph 50 has an x-axis 51 showing a progression in time in hours and callouts showing days. The y-axis 52 shows electrical sensitivity measured in nA/mg/dL.

The graph 50 has measurements for seven different sensors. A first set of sensors 53 includes three sensors where each sensor has an enzyme layer without a crosslinker. Put another way, the enzyme layer of each of the first set of sensors 53 has no enzyme immobilization network. A second set of sensors 54 includes four sensors. Each of the sensors of the second set of sensors 54 has an enzyme layer with a crosslinker (also known as a crosslinking agent), such that the enzyme layer has the enzyme immobilization network. The second set of sensors 54 used an enzyme formulation of 1) GOx as the enzyme, 2) a polymer being an aqueous polyurethane dispersion with polyvinylpyrrolidone, and 3) polyethylene glycol dialdehyde as the crosslinker. The samples, or the first set of sensors 53 and the second set of sensors 54 embodies the wire, the enzyme layer (with or without the crosslinker) and an outer layer to test functionality over a duration. By having an outer layer, the sensitivity may be measured in the range of 0 nA/mg/dL to 0.080 nA/mg/dL.

Generally, the sensitivity for all of the seven sensors start near the same range at day zero, and the first set of sensors 53 (each without the crosslinker) generally declined steadily in sensitivity over time. As illustrated, sensitivity of the first set of sensors 53 dropped considerably within 14 days, and showed dramatic loss of sensitivity within five days. In contrast, the second set of sensors 54 (each with the crosslinker) showed improved sensitivity in the first 200 hours, and then continued with exceptional sensitivity up to 21 days, when the test was stopped. Not only did the second set of sensors 54 having the crosslinker exhibit improved sensitivity over the first set of sensors 53 without the crosslinker, they also had improved stability and exhibited better linearity over the full 21 days. With better sensitivity, significantly longer stability (e.g., electrical sensitivity remaining stable for over 21 days), and improved linearity, the second set of sensors 54 have a much longer useful life in a patient while requiring fewer replacements and fewer or no local calibrations.

FIG. 5B is a chart showing sensitivity results for sensors, in accordance with some embodiments. A summary chart 55 shows actual results of the improvement to the stability over 21 days on account of the enzyme layer having the crosslinker or the enzyme immobilization network. The samples embody the wire and the enzyme layer (with and without the crosslinker) to test the performance of the enzyme layer without a limiting layer barrier. Thus, the sensitivity readings are in the range of 0 nA/mg/dL to 30 nA/mg/dL. The chart 55 has a y-axis 56 that shows sensitivity measured in nA/mg/dL. The x-axis shows data for the first set of sensors 53 without the crosslinker and the second set of sensors 54 with the crosslinker. A first bar 57 represents measurements from the first set of sensors 53 without the crosslinker of FIG. 5A, and a second bar 58 represents measurements from the second set of sensors 54 having the crosslinker of FIG. 5A. As illustrated, the first set of sensors 53 without crosslinkers in the first bar 57 showed an average sensitivity over the 21 day test period of about 10 nA/mg/dL, while the second set of sensors 54 with crosslinkers in the second bar 58 show an average sensitivity of about 22 nA/mg/dL. Accordingly, the use of a crosslinker to form an enzyme immobilization network more than doubled the sensitivity of the enzyme layer without any crosslinker.

FIG. 5C is a graph showing sensitivity results for a continuous glucose monitor, in accordance with some embodiments. A graph 70 shows an actual accelerated aging test performed on sensors. The samples were sterilized pre-test. The y-axis shows a sensitivity current measured in microamps, while the x-axis shows time in hours. The test was performed on the sensors at approximately 50° C. in order to mimic aging of the sensor and enzyme over time. Using high temperature heating on the sensor as a way to predict aging performance, is well known and well established in the art. A bottom line 71 in graph 70 shows sensitivity for a first set of sensors 73 having no crosslinker, and therefore lack the enzyme immobilization network. Generally, the sensitivity decreases from 0.025 μA to nearly 0 μA by 20,000 seconds or in about 5.6 hours. In contrast, the top line 72 shows sensitivity for a second set of sensors 74 with the crosslinker having the enzyme immobilization network. The sensitivity at the beginning of the test is approximately 0.038 μA and decreases only slightly to about 0.035 μA by 20,000 seconds or in about 5.6 hours.

This test illustrates two very important features. First, the second set of sensors 74 with the crosslinker and enzyme immobilization network have incredibly stable sensitivity over the entire period of the aging test. Second, the second set of sensors 74 with the crosslinker exhibit nearly double the sensitivity of the first set of sensors 73 without the crosslinker at the beginning of the test, and the relative superiority of the second set of sensors 74 with the crosslinker increases as time progresses.

FIG. 6 is a graph showing sensitivity results for a continuous glucose monitor, in accordance with some embodiments. It is well established with known prior art sensors that sterilizing CGM sensors with gas, such as EtO gas, results in a degradation of sensitivity and stability. A graph 60 illustrates that sterilizing a sensor which incorporates the enzyme immobilization network actually removes all negative effects of gas sterilization, and has been tested and measured to show an improvement in sensitivity after gas sterilization. Referring now to the graph 60, the y-axis 61 shows sensitivity measured in nA/mg/dL. The x-axis shows sensitivity data for 10 different sensors. A first set of five sensors 62, numbered 1 through 5, indicate sensors that do not have the crosslinker, and therefore do not have the enzyme immobilization network. A second set of sensors 63, numbered 6 through 10, indicate sensors that do have the crosslinker, and therefore have the enzyme immobilization network in their enzyme layer. Each of the sensors, 1 through 10, have two informational data bars. The left bar 64 for each sensor indicates the sensitivity for that sensor after manufacturing has been completed, but prior to gas sterilization with EtO gas (e.g., pre-EtO). The right bar 65 for each sensor indicates the sensitivity for that sensor after that sensor has been sterilized with EtO gas (e.g., post-EtO).

As illustrated for each of the sensors 1 through 5 of the first set of sensors 62, which do not have the crosslinker, sterilizing the sensor with EtO gas substantially diminished sensitivity as observed by the decrease in sensitivity when comparing each left bar 64 to each right bar 65 for each sensor, 1 through 5. Line 68 shows the percent change in sensitivity from the pre-sterilization data of left bars 64 to the post sterilization data of right bars 65 for each of the sensors 1 through 10. The y-axis 69, on the right-hand side of the graph 60, indicates the change in sensitivity in percent. For sensors 2, 3, and 4, the sensitivity decreased nearly in half, while in sensor 1 sensitivity decreased by nearly two thirds. Sensor 5 showed a decrease of over one third. On average, sterilizing the sensors 1 through 5, which do not have the crosslinker, decreased sensitivity by about an average of 50%. In sharp contrast, sensors 6 through 10 of the second set of sensors 63, show that EtO gas sterilization not only did not degrade sensitivity, but actually improved sensitivity from about 2% to over 10%. This is illustrated in graph 60 by viewing line 68 and by comparing the left bars 64 to the right bars 65 for each sensor, 6 through 10. In this way, the addition of the crosslinker, which provided the support in the formation of the enzyme immobilization network, enabled improved sensitivity for every tested sensor post gas sterilization.

In some embodiments, the continuous glucose sensor with the enzyme immobilization network (e.g., crosslinker) has a first measured electrical enzyme sensitivity prior to gas sterilization and a second measured electrical enzyme sensitivity after the gas sterilization. The gas sterilization may be by ethylene oxide (EtO) sterilization. When comparing the first measured electrical enzyme sensitivity to the second measured electrical enzyme sensitivity, the second measured electrical enzyme sensitivity is greater than the first measured electrical enzyme sensitivity as evidenced in FIG. 6, graph 60 (see the second set of sensors 63 with crosslinkers data).

Referring to FIG. 3, the interference membrane 34 is layered between the electrical conductive wire 33 and the enzyme membrane 35 (also known as enzyme layer) in working electrode 31. Generally, the interference membrane 34 is applied as a monomer, with selected additives, and then polymerized. The interference membrane 34 may be electrodeposited onto the electrical conductive wire 33 in a very consistent and conformal way, thus reducing manufacturing costs as well as providing a more controllable and repeatable layer formation. The interference membrane 34 is nonconducting of electrons, but may pass ions and hydrogen peroxide at a preselected rate. Further, the interference membrane 34 may be formulated to be permselective for particular molecules. In one example, the interference membrane 34 is formulated and deposited in a way to restrict the passage of active molecules, which may act as contaminants to degrade the conductive wire 33, or that may interfere with the electrical detection and transmission processes.

In some embodiments, the interference membrane 34 is nonconductive of electrons, but is conductive of ions. In practice, a particularly effective interference membrane may be constructed using, for example, Poly-Ortho-Aminophenol (POAP). POAP may be deposited onto the conductive wire 33 using an electrodeposition process, at a thickness that can be precisely controlled to enable a predictable level of hydrogen peroxide to pass through the interference membrane 34 to the conductive wire 33. Further, the pH level of the POAP may be adjusted to set a desirable permselectivity for the interference membrane 34. For example, the pH may be advantageously adjusted to significantly block the passage of larger molecules such as acetaminophen, thereby reducing contaminants that can reach the conductive wire 33. It will be understood that other materials may be used. For example, the interference layer may include a polymer that has been electropolymerized selected from polyaniline, naphthol or phenylenediamine, 2-aminophenol, 3-aminophenol, 4-aminophenol, m-phenylenediamine, o-phenylenediamine, p-phenylenediamine, pyrrole, derivatized pyrrole, aminophenylboronic acid, thiophene, porphyrin, aniline, phenol, thiophenol, or blends thereof.

When the working electrode 31 is exposed to EtO gas, the EtO gas passes through the glucose limiting layer 36 (if present) and contacts and may penetrate the enzyme membrane 35. However, the immobilization network in the enzyme membrane 35 resists the negative effect of the EtO gas, and acts to improve the stability and sensitivity of the resulting biological sensor. Further protection may be provided as the interference membrane 34 may act as a physical shield to reduce the level of EtO passing through the enzyme layer that can reach the conductive wire 33, thereby reducing the negative oxidation effects of the EtO.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed, is:

1. A working wire of a continuous biological sensor, comprising:
a substrate for the sensor, the substrate having a conductive surface;
an enzyme layer on the conductive surface comprising:
an enzyme;
an immobilization matrix; and
an enzyme immobilization network formed using a polymeric crosslinking agent and a non-polymeric crosslinking agent crosslinking the enzyme and the immobilization matrix, wherein the polymeric crosslinking agent and the non-polymeric crosslinking agent are a combination of polyethylene glycol (PEG) dialdehyde and glutaraldehyde; and
a protective layer over the enzyme layer.

2. The working wire of claim 1, wherein the immobilization matrix is a polymer selected from polyurethane (PU), polyacrylic acid, polyacrylamide, polyvinylpyrrolidone (PVP), polyethylene glycol (PEG), or polyvinyl alcohol (PA) and its copolymers, or copolymers of N-(2-hydroxypropyl)-methacrylamide, polydimethylsiloxane (PDMS), polyamides, polyacrylates, polyethylene, polycarbonates, or combinations thereof.

3. The working wire of claim 1, wherein the immobilization matrix is a protein selected from a bovine serum albumin (BSA), human serum albumin (HSA), carboxymethyl cellulose (CMC), collagen, or combinations thereof.

4. The working wire of claim 1, wherein the enzyme immobilization network stabilizes a glucose sensitivity of the sensor over time.

5. The working wire of claim 1, wherein the polyethylene glycol (PEG) dialdehyde is configured to allow the enzyme to rotate around crosslinked bonds.

6. The working wire of claim 1, wherein the glutaraldehyde is configured to immobilize the enzyme, thereby stabilizing a glucose sensitivity of the sensor over time.

7. The working wire of claim 1, wherein the enzyme is glucose oxidase (GOx).

8. The working wire of claim 1, wherein the protective layer is a glucose limiting layer configured to restrict passage of glucose molecules into the enzyme layer.

9. The working wire of claim 1, wherein the sensor has a first measured electrical enzyme sensitivity prior to gas sterilization, a second measured electrical enzyme sensitivity after the gas sterilization, and the second measured electrical enzyme sensitivity is greater than the first measured electrical enzyme sensitivity.

10. The working wire of claim 1, wherein the enzyme immobilization network is configured to resist degradation caused by ethylene oxide (EtO) gas during sterilization.

11. A method of manufacturing a working wire of a continuous biological sensor, comprising:
providing a substrate for the sensor, the substrate having a conductive surface;
forming an enzyme layer on the conductive surface, the forming comprising:
combining an enzyme with a solvent creating an enzyme mixture;
mixing an immobilization matrix with the enzyme mixture;
combining a polymeric crosslinking agent and a non-polymeric crosslinking agent with the enzyme mixture and the immobilization matrix creating a crosslinked mixture, wherein the polymeric crosslinking agent and the non-polymeric crosslinking agent are a combination of polyethylene glycol (PEG) dialdehyde and glutaraldehyde, to form an enzyme immobilization network that crosslinks the enzyme and the immobilization matrix; and
applying the crosslinked mixture to the substrate;
curing the crosslinked mixture on the substrate, after the applying; and
forming a protective layer over the enzyme layer.

12. The method of claim 11, further comprising allowing the crosslinked mixture to stabilize before applying the crosslinked mixture to the substrate.

13. The method of claim 11, wherein the immobilization matrix is a polymer selected from polyurethane (PU), polyacrylic acid, polyacrylamide, polyvinylpyrrolidone (PVP), polyethylene glycol (PEG), or polyvinyl alcohol (PA) and its copolymers, or copolymers of N-(2-hydroxypropyl)-methacrylamide, polydimethylsiloxane (PDMS), polyamides, polyacrylates, polyethylene, polycarbonates, or combinations thereof.

14. The method of claim 11, wherein the immobilization matrix is a protein selected from a bovine serum albumin (BSA), human serum albumin (HSA), carboxymethyl cellulose (CMC), collagen, or combinations thereof.

15. The method of claim 11, wherein the enzyme immobilization network stabilizes a glucose sensitivity of the sensor over time.

16. The method of claim 11, wherein the polyethylene glycol (PEG) dialdehyde is configured to allow the enzyme to rotate around crosslinked bonds.

17. The method of claim 11, wherein the glutaraldehyde is configured to immobilize the enzyme, thereby stabilizing a glucose sensitivity of the sensor over time.

18. The method of claim 11, wherein the mixing comprises high shear mixing.

19. The method of claim 11, wherein the enzyme is glucose oxidase (GOx).
